(19) United States
(12) Patent Application Publication
(10) Pub. No.: US 2004/0192994 A1
(43) Pub. Date: Sep. 30, 2004

(54) Propylene production

(76) Inventors: Robert S. Bridges, Friendswood, TX (US); Donald H. Powers, Pearland, TX (US); Steven T. Coleman, Humble, TX (US)

Correspondence Address: LYONDELL CHEMICAL COMPANY, 3801 WEST CHESTER PIKE, NEWTOWN SQUARE, PA, 19073, US

(21) Appl. No.: 10/401,166

(22) Filed: Mar. 26, 2003

(51) Int. Cl. C07C 6/00; C07C 5/23; C07C 5/25

(52) U.S. Cl. 585/664

(57) ABSTRACT

A method for making propylene from alpha olefins, internal linear olefins, and isoolefins wherein the internal linear olefins are separated and then disproportionated with ethylene to form a propylene product, while the alpha olefins are subjected to double bond isomerization to form additional internal linear olefins, and the isoolefins are subjected to skeletal isomerization to form yet additional internal linear olefins.

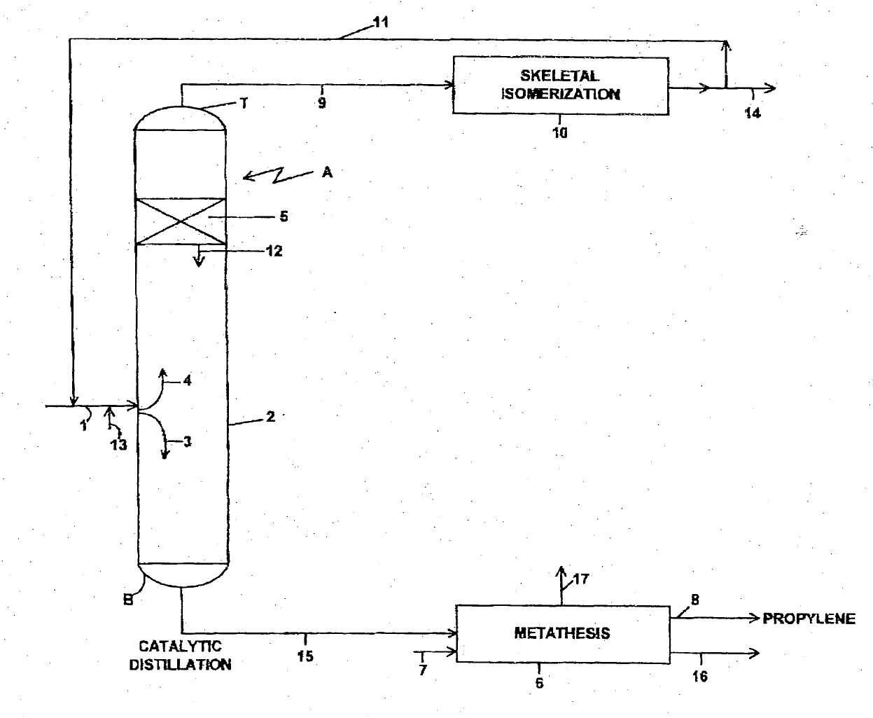

### BACKGROUND OF THE INVENTION

1. Field of the Invention

This invention relates to a method for making propylene utilizing hydrocarbons that have four carbon atoms per molecule (C₄'s). More particularly this invention relates to a process for forming propylene from an isobutylene containing feedstock.

2. Description of the Prior Art

Although this invention will, for the sake of clarity of understanding, be described in the context of an olefin production plant (olefin plant), this invention is broadly applicable to the use, as feed thereto, of any hydrocarbon stream containing the requisite feed component(s) as described in detail herein.

Thermal cracking of hydrocarbons is a petrochemical process that is widely used to produce olefins such as ethylene, propylene, butenes, butadiene, and aromatics such as benzene, toluene, and xylene. In an olefin plant, a hydrocarbon feedstock such as naphtha, gas oil, or other fractions of whole crude oil is mixed with steam which serves as a diluent to keep hydrocarbon molecules separated. This mixture, after preheating, is subjected to severe hydrocarbon thermal cracking at elevated temperatures (1450° F. to 1550° F.) in a pyrolysis furnace (steam cracker).

The cracked effluent product from the pyrolysis furnace contains gaseous hydrocarbons of great variety (from 1 to 35 carbon atoms per molecule). This effluent contains hydrocarbons that are aliphatic, aromatic, saturated, and unsaturated, and can contain significant amounts of molecular hydrogen (hydrogen).

The cracked product of a pyrolysis furnace is then further processed in the olefin plant to produce, as products of the plant, various separate individual product streams of high purity such as hydrogen, ethylene, propylene, mixed hydrocarbons having four carbon atoms per molecule (crude C₄'s), and pyrolysis gasoline. It is this crude C₄ product of the debutanizer of an olefin plant upon which is focused this description of one embodiment within this invention.

Crude C₄'s can contain varying amounts of n-butane (butane), isobutane, butene-1, butene-2 (cis- and/or trans-), isobutylene (isobutene), acetylenes, butadiene, and molecular hydrogen (hydrogen). The term butene-2 as used herein includes cis-butene-2, trans-butene-2, or a mixture of both.

Heretofore crude C₄'s have been subjected to butadiene extraction or butadiene selective hydrogenation to remove most, if not essentially all, of the butadiene and acetylenes present. Thereafter the crude C₄ raffinate was subjected to an etherification process wherein the isobutylene was converted to methyl tertiary butyl ether (MTBE).

Also heretofore crude C₄'s have been subjected to selective hydrogenation of dioelfins (butadiene) with simultaneous isomerization of alpha olefins (butene-1) to internal olefins (butene-2) followed by etherification of the isoolefins (isobutylene to MTBE), and finally metathesis of internal olefins (butene-2) with ethylene to produce propylene, see U.S. Pat. No. 5,898,091 to Chodorge et al.

In addition, catalytic distillation of various hydrocarbon streams for various purposes such as hydrogenation, mono-olefin isomerization, etherification, dimerization, hydration, dissociation, and aromatic alkylation has been disclosed, see U.S. Pat. No. 6,495,732 B1.

If MTBE market demand should decline, it is desirable to be able to utilize the isobutylene that was formerly used in producing MTBE to produce a different product that is enjoying more robust market demand.

It has been suggested that the isobutylene be dimerized to iso-octene followed by hydrogenation to iso-octane, or be alkylated to iso-octane, neither of which promises to be a cost-effective solution.

### SUMMARY OF THE INVENTION

In accordance with this invention, the isoolefins and internal olefins in a feedstock are converted into propylene.

In the context of an olefin plant, this increases the output of propylene product from that plant, a distinct advantage in a robust propylene market.

Another advantage for this invention is that in addition to producing more propylene product, this invention produces, at the same time, a C₅ plus olefin product that boils in the automotive gasoline range, and, therefore, is a valuable addition to the overall gasoline pool.

### DESCRIPTION OF THE DRAWING

The drawing shows a schematic flow scheme of one embodiment within this invention.

### DETAILED DESCRIPTION OF THE INVENTION

By this invention, and referring to the drawing, a feedstock 1 containing in whole or in part a first mixture composed of alpha olefins, internal olefins, and isoolefins having four carbon atoms per molecule, is subjected to a catalytic distillation operation A wherein distillation conditions coupled with double bond isomerization conditions separate the internal olefins 3 from the alpha olefins and isoolefins 4 in tower 2, and convert alpha olefins to internal olefins in zone 5. The internal olefins 3 are subjected to disproportionation conditions (metathesis) in zone 6 in the presence of an ethylene co-feed 7 to form the desired propylene product 8. The alpha olefins and isoolefins are separately recovered in line 9 and subjected to skeletal isomerization conditions in zone 10 which convert (transform) isoolefins at least in apart to linear internal olefins, e.g., butene-2 (sometimes referred to as “internal olefins”). The internal olefins thus formed are returned by way of line 11 along with unconverted isoolefins and any alpha olefins present, back to the catalytic distillation zone A as feed or co-feed for the catalytic distillation operation aforesaid.

The feedstock (feed) for this invention can be any suitable stream that contains the requisite C₄ components. One such stream is a high purity isobutylene stream that contains no n-butane or isobutane. Such a stream can be obtained as a byproduct of a propylene oxide production process. This feed, with or without added hydrogen, is passed into catalytic distillation zone A which is a combination of a conventional distillation tower (column) 2 and a double bond isomerization zone (reactor) 5 carried internally of tower 2 so that the feed components, upon entering the tower are subjected in a single vessel both to conventional (simple) distillation separation conditions in tower 2, and double bond isomerization conditions in isomerization zone 5.

Other suitable feeds include raffinate products obtained from the extractive distillation of crude C₄'s with a solvent that preferentially removes diolefins such as butadiene by altering the relative volatility of the butadiene. These raffinate products (sometimes called raff-1) are depleted (less than about 1 weight percent (wt. %), based on the total weight of the raffinate) in butadiene content, but rich (over 75 wt. % on the same basis) in a mixture of butene-1, butene-2, and isobutylene—all desired components for the process of this invention. The small amount of butadiene left in a raffinate product can be readily hydrogenated in zone 5 of tower 2. Another suitable feed is a crude C₄ stream from an olefin plant debutanizer as described herein. This stream requires conventional hydrogenation of its butadiene content either before it is passed into tower 2 or during the process of this invention, e.g., downstream of tower 2 and upstream of zone 10 in conventional hydrogenation equipment (not shown). This is so because the butadiene content of the feed to zone 10 is preferably less than 100 parts per million (ppm).

It is known in the art to process olefins using catalytic distillation techniques. For example, alpha olefins have heretofore been oligomerized using catalytic distillation techniques, see U.S. Pat. No. 4,935,577 to Huss, Jr., et al. The isomerization catalyst in zone 5 is fitted into a distillation tower 2 that is equipped with an overhead cooler, condenser, and reflux pump, internal stages such as fractionation trays, a reboiler, and standard control instrumentation which are not shown in the drawing for simplicity sake since all are well known in the art.

Depending on the boiling range of the feed, which can vary considerably, the feed is introduced into the interior of tower 2 in a manner such that at least the component of the feed that is to be isomerized in zone 5 travels into contact with the catalyst bed(s) in that zone. Thus, depending on the feed composition and other variables such as the distillation conditions, the feed can be introduced above or below zone 5. Preferably, the feed is introduced below zone 5 so that under the prevailing distillation conditions higher boiling components such as butane and internal olefins (butene-2) are recovered as shown by arrow 3 at the tower bottom B for recovery therefrom, while lower boiling components such as isobutane, isoolefins (isobutylene), alpha olefins (butene-1), and diolefins (butadiene) are recovered as shown by arrow 4-at the tower top T and, in the process of such upward travel, into contact with one or more double bond isomerization catalyst beds in zone 5 before reaching the tower top.

The prevailing distillation conditions within tower 2 are distributed throughout the internal height of such tower so that not only is the desired distillation separation accomplished, but also the temperature and pressure conditions in tower 2 in the location of the catalyst in zone 5 favor the double bond isomerization conditions required for that particular catalyst (or catalysts).

In the double bond isomerization reactor 5 in tower 2, alpha-olefins such as butene-1 are isomerized to internal olefins up to their chemical equilibrium constraint. Newly formed internal olefins 12 are then refluxed down out of zone 5 to the tower bottom B for recovery, thus increasing the internal olefin content over that of the original feed. This is most advantageous in increasing the amount of desired propylene product recovered from the process of this invention. The double bond isomerization catalyst used in this invention can also serve to promote the hydrogenation of dioolefins and acetylenics if hydrogen is present. Hydrogen 13 can be added to the feed for butadiene hydrogenation purposes up to about 1 wt. % based on the total weight of the feed, greater amounts of hydrogen tending to reduce the selectivity of the catalyst for butadiene hydrogenation.

Thus, if diolefins and/or acetylenes (and hydrogen) are present in the feed they will be hydrogenated in zone 5 while the double bond isomerization is taking place. For example, butadiene will be converted to an equilibrium mixture of butene-1 and butene-2 which is also advantageous in increasing the ultimate propylene product yield for the process of this invention. Since higher boiling internal olefins 12 formed in isomerization zone 5 are refluxed downwardly toward the tower bottom for recovery therefrom, a second mixture composed of lower boiling isobutane, unconverted alpha olefins, and isoolefins travels upwardly out of catalyst zone 5 to the tower top for recovery by way of line 9 from the tower top. Contrary to the ease of separating butene-2 from butene-1 by simple distillation, butene-1 and isobutylene cannot be economically separated by simple distillation because their boiling points differ by only 0.6° C.

The overhead from the top of tower 2 is passed by way of line 9 to skeletal isomerization zone 10 which converts isoolefins to an equilibrium mixture of alpha and linear internal monoolefins, e.g., isobutylene to a mixture of butene-1 and butene-2. The product of this zone is a third mixture found in line 11 which is composed of alpha olefins, linear internal olefins, unconverted isoolefins, and isobutane. This third mixture is returned by way of line 11 as feed or co-feed to catalytic distillation zone A for the removal of the linear internal olefins to the tower bottom, isomerization of alpha olefins to yet additional linear internal olefins, and ultimate recycling of unconverted isoolefin in the aforesaid second mixture (from the top of tower 2) to zone 10 by way of line 9.

It should be noted here that in this invention, skeletal isomerization in zone 10 is distinguished from double bond isomerization in zone 5 in that skeletal isomerization involves the movement of a carbon atom to a new location on the carbon atom skeleton of the molecule, e.g., from a branched isobutylene skeleton to a linear or straight chain (not branched) butene skeleton. Double bond isomerization does not involve movement or shifting of carbon atoms on the skeleton, but rather only involves movement of a double bond within the skeleton while the carbon atoms that form the carbon atom skeleton remain in their original locations in that skeleton.

Since isobutane, if it is present in the feed stream, is unreactive it will build up in the third mixture product in line 11 of the skeletal isomerization zone 10. Accordingly, a purge stream 14 can be taken from this third mixture product to relieve isobutane buildup and can be employed elsewhere. For example, since this purge stream contains isobutane, alpha olefins, and internal olefins, it could be employed to advantage in an alkylation process to form a gasoline grade alkylate of mixed isooctanes, plus a separate butane product.

Normal butane and internal linear olefins are recovered at the bottom of tower 2 as shown by arrow 3 for recovery therefrom by way of line 15, and for use as feed for metathesis zone 6. In zone 6 butene-2 and ethylene co-feeds are metathesised to propylene product, 1 mole of butene-2 and 1 mole of ethylene yielding 2 moles of propylene.

The metathesis reaction yields useful byproducts. One such byproduct is a mixed olefinic stream 16 that contains hydrocarbons of 5 carbon atoms per molecule and heavier that are in the automotive gasoline boiling range (from about 77° F. to about 437° F.). For example, this byproduct can contain a substantial majority (up to 80 wt. % or more) C₅ olefins, and a significant minority (up to 20 wt. %) C₆ and heavier olefins, all wt. % based on the total weight of the byproduct.

Normal butane, if present in the feed, being unreactive in the process of this invention, tends to build up in the system. Stream 17 can be taken from the process, in whole or in part, as a useful byproduct thereof in that it contains butene-2 as well as n-butane, and can be used in alkylation to form gasoline grade alkylate of mixed isooctanes plus a separate butane product. This helps relieve n-butane build-up in the system caused by recycle of the unreacted butanes within the metathesis process. Alternatively, stream 17 can, in whole or in part, be recycled as co-feed to tower 2 such as by introduction into stream 1 and/or metathesis zone 6 such as by introduction into stream 15. If stream 17 is recycled as an additional source of butene-2 for zone 6, a purge stream (not shown) can be taken from stream 17 and removed from the process in order to relieve n-butane build-up in the system. This way, butene-2 that was not disproportionated with ethylene in its first pass through zone 6 is returned to the process for another pass through that zone.

The distillation conditions in tower 2 will be broader than the double bond isomerization conditions required by the isomerization catalyst in zone 5, but will encompass those isomerization conditions so that at least one catalyst bed can be fixed in zone 5 in the interior of tower 2 in a temperature and pressure environment that meets the double bond isomerization reaction (and butadiene hydrogenation) temperature and pressure requisites for that particular catalyst. Thus, the catalyst can be located anywhere along the internal height of the tower where the desired isomerization conditions are present.

A given catalyst could be located centrally of the tower height, or nearer the top of the tower, or nearer the bottom of the tower depending on where in the tower the combination of temperature and pressure conditions best match the necessary isomerization (and hydrogenation) conditions.

The tower distillation temperature range from tower bottom to tower top will vary widely depending upon the operating pressure of the tower and, to a lesser extent the relative proportion of the C₄'s in feed 1. An increase in pressure in the tower provides a higher temperature in the tower for the isomerization reaction. In establishing tower distillation conditions, the starting point is the desired component separation, e.g., the separation by simple distillation of butene-1 from butene-2. The top temperature and pressure of the tower is then set to effect the desired separation by simple distillation. The rate of reflux and number of trays is then set. The higher the reflux rates, the fewer the number of trays are needed, and the lower the pressure drop through the tower. The less the reflux, the more trays are needed and the higher the pressure drop through the tower. The distillation conditions of temperature and pressure can vary widely, and one skilled in the art would readily know how to determine the appropriate distillation conditions.

Once the tower top temperature and pressure is set along with the reflux rate and number of trays, the tower bottom temperature and pressure is determined. For a separation by simple distillation of butene-1 from butene-2, the tower top temperature will range from about 20° F. to about 260° F. at pressures ranging from about 0 psig to about 400 psig. It is presently preferred that the distillation conditions (and tower design) be set so that they encompass the double bond isomerization conditions in a manner such that the feed can be introduced into the tower below the isomerization catalyst as shown in the drawing, and, for example, isobutylene, butene-1, and butadiene if present, rise upwardly, arrow 4, into contact with the catalyst in zone 5, while butene-2 is separated from the butene-1 etc. and, along with any n-butane present, travels downwardly, arrow 3, toward the tower bottom.

The double bond isomerization conditions within catalytic distillation zone A where the double bond isomerization zone (and catalyst) 5 are located are a temperature of from about 70° F. to about 270° F. and a pressure of from about 20 psig to about 400 psig.

The catalyst used in zone 5 can vary widely. If there is no appreciable (less than about 100 ppm) butadiene content in feed 1, the catalyst can be primarily only an isomerization component and need not contain a component (catalyst) that promotes the hydrogenation of butadiene. If there is appreciable butadiene content in the feed (greater than about 100 ppm and up to about 1 wt. % based on the total weight of the feed), the catalyst can additionally contain a hydrogenation component. If butadiene is present in the feed, the isomerization catalyst and hydrogenation catalyst can be employed in zone 5 either mixed with one another in a single catalyst bed in zone 5, or in separate discrete catalyst beds in zone 5, each discrete bed containing either solely isomerization catalyst or solely hydrogenation catalyst, or a dual function catalyst as explained herein below.

The isomerization catalyst employed in zone 5 favors the formation of internal linear olefins over the formation of linear alpha olefins. Generally, the catalysts employed promote, preferably primarily promote, double bond shifts within a specific olefin molecule (double bond isomerization), so any such catalyst that is suitable for use in a catalytic distillation process can be used. The catalysts useful in this invention will be obvious to one skilled in the art since they are either commercially available or fully disclosed in the prior art. Such catalysts include acidic ion exchange resins such as sulfonated resins with sulfonic acid sites (U.S. Pat. No. 3,326,866), perfluorinated polymer sulfonic acid catalyst, phosphoric acid catalyst, carboxylic acid catalyst, fluorinated alkyl sulfonic acid catalyst, alumina plus alkali metal (U.S. Pat. No. 4,992,612), zinc aluminate (U.S. Pat. No. 4,692,430), zirconia, sulfated zirconia, cobalt/sulfur catalyst (U.S. Pat. No. 3,962,367), ruthenium oxide (U.S. Pat. No. 4,962,267), alumino phosphates, and zeolite structures with or without alkali metal (U.S. Pat. No. 4,992,613), and alumina or silica alumina.

The hydrogenation catalyst component employed in zone 5 favors the saturation with hydrogen of at least one double bond in a butadiene molecule. These catalysts will also be obvious to one skilled in the art and commercially available. Such catalysts can contain a noble metal, e.g., at least one of palladium, platinum, and rhodium either supported or unsupported. When the noble metal is supported on an acidic material, a combined function catalyst of double bond isomerization and hydrogenation is achieved. Suitable catalysts having both a double bond isomerization and a hydrogenation capability will be obvious to those skilled in the art, and include, but are not limited to, at least one of palladium, platinum, and rhodium carried on an acidic support such as alumina, silica alumina, and the like. The noble metal(s) can be present in the catalyst in amounts of from about 0.1 to about 0.3 wt. % based on the total weight of the catalyst. For other suitable catalysts for both hydrogenation and double bond isomerization, see U.S. Pat. Nos. 5,955,397 and 6,495,732 B1.

The metathesis zone 6 operating conditions can vary widely, but are generally a temperature of from about 300° F. to about 800° F., a pressure of from about 200 psig to about 600 psig, and a weight hourly space velocity of from about 1.0 h⁻¹ to about 100 h⁻¹.

Suitable catalysts that promote, preferably primarily promote, metathesis as described herein are known in the art, and include at least one of halides, oxides and/or carbonyls of at least one of molybdenum, tungsten, rhenium and/or magnesium carried on a support such as silica and the like. The conversion of butene-2 in the presence of excess ethylene to propylene is known and has been demonstrated; see R. L. Banks, Journal of Molecular Catalysis, Vol. 8, p. 269-276, 1980, ISSN 0304-5102. For more information on is olefin metathesis, see Discovery and Development of Olefin Disproportionation (Metathesis) by Robert L. Banks, American Chemical Society Symposium Series, No. 222, Heterogeneous Catalysis: Selected American Histories, B. H. Davis and W. P. Hettinger, Jr., Editors, American Chemical Society, 1983, ISSN 0097-6156.

The skeletal isomerization zone 10 operating conditions also vary widely, but generally are a temperature of from about 450° F. to about 1200° F., a pressure of from about 0 psig to about 150 psig, and a weight hourly space velocity of from about 1.0 h⁻¹ to about 50 h⁻¹. Skeletal isomerization catalysts useful in this invention are known in the art and include zeolites having one-dimensional pore structures with a pore size ranging from greater than about 0.42 nanometers (nm) and less than about 0.7 nm. This type of isomerization process is known, see U.S. Pat. No. 6,111,160 to Powers et al., and U.S. Pat. No. 6,323,384 also to Powers et al.

### EXAMPLE

A crude C₄ stream from an olefin plant debutanizer consisting essentially of about 2 wt. % isobutane, about 24 wt. % isobutylene, about 17 wt. % butene-1, about 10 wt. % butene-2, about 40 wt. % butadiene, and about 7 wt. % n-butane, all wt. % based on the total weight of the stream, is employed as feed to a catalytic distillation tower configured as described above and shown in the drawing with a double bond isomerization/hydrogenation catalyst bed carried in the tower in the location shown in the drawing. The double bond isomerization/hydrogenation catalyst is a commercially available catalyst composed of 0.2 wt. %, based on the total weight of the catalyst, of palladium on a silica/alumina support. The catalytic distillation conditions are set to fractionally distill butene-2 from butene-1 so that butene-1 will rise, arrow 4, into contact with the double bond isomerization catalyst in zone 5 of the drawing and butene-2 will pass downwardly, arrow 3, to the bottom of the distillation tower for recovery therefrom. Under such distillation conditions, isobutane, isobutylene, butadiene, and hydrogen rise with butene-1 into contact with the catalyst in zone 5, while butane travels downwardly with the butene-2.

The distillation conditions were set with a tower top temperature and pressure of about 116° F., and about 65 psig, a reflux ratio of about 10, and 100 valve trays in the tower, which yields a tower bottom temperature of about 140° F. and pressure of about 80 psig.

These distillation conditions provide isomerization conditions in zone 5 of about 123° F. at a pressure of about 70 psig.

A mixture of n-butane and butene-2 containing about 60 wt. % butene-2, based on the total weight of the mixture, is removed from the tower bottom and passed by line 15 to metathesis zone 6 wherein it is contacted with a molar excess of ethylene in the presence of a commercially available metathesis catalyst composed of a mixture of tungsten oxide and magnesium oxide at a temperature of about 550° F. and pressure of about 300 psig. Two products are recovered from zone 6, a propylene stream at 8, and a separate gasoline grade stream at 16 containing about 80 wt. % C₅ olefins based on the total weight of the stream with the remainder being essentially C₆ and higher olefins boiling within the automotive gasoline range aforesaid.

In zone 5, butene-1 is transformed to butene-2, and the newly formed butene-2, due to the prevailing distillation conditions, is refluxed downwardly, arrow 12, out of zone 5 for collection in and recovery from the tower bottom as additional feed material for the metathesis reaction zone 6.

Butadiene is selectively hydrogenated in zone 5 to a mixture of butene-1 and butene-2, the newly formed butene-2 being refluxed downwardly, arrow 12, 10 as described above, for use as metathesis feed.

Newly formed butene-1 from butadiene hydrogenation, existing butene-1 that was not converted to butene-2, isobutane, and isobutylene, all in zone 5, rise under the prevailing distillation conditions to the tower top where they are collected and transferred by way of line 9 to skeletal isomerization zone 10.

In zone 10 the primary reaction is the transformation of branched isobutylene to linear butenes (-1 and -2). A secondary reaction is double bond isomerization of butene-1 to butene-2. Accordingly, the product of zone 10 is a mixture of butene-2 (newly formed and unconverted), butene-1, n-butane, unconverted isobutylene, and isobutane. This product is recycled as co-feed to the catalytic distillation zone A to make yet more butene-2 from butene-1 (in zone 5), and again separate existing and newly formed butene-2 for use in the metathesis operation for producing additional propylene product. The isobutylene in this recycle stream 11 ultimately reaches skeletal isomerization zone 10 again at which time at least part of it is converted to a mixture of butenes.

On a single pass basis, and with no recycle, about 65 wt. percent of the butene-2 in stream 15 is converted to either propylene or gasoline, with about 90 percent selectivity to propylene. In the skeletal isomerization operation, also on a single pass basis, there is about a 50 wt. percent conversion of isobutylene with a 90 percent selectivity to butene-1 and butene-2.

It can be seen from the foregoing example that the process of this invention is replete with opportunities throughout the entire process to make newly formed butene-2 which is then made into the desired propylene product. Accordingly, it can be seen that the process of this invention is highly leveraged toward and very efficient in making new butene-2. Thus, in addition to making propylene out of the butene-2 originally in the feedstock to the process, this invention significantly increases the amount of propylene product obtained from a given feed based on the butene-2 content of the original feed.

Reasonable variations and modifications are possible within the scope of this disclosure without departing from the spirit and scope of this invention.

We claim:

1. A method for forming propylene comprising providing a feedstock containing at least in part one of (A) a first mixture of hydrocarbons comprising alpha olefins, internal linear olefins, and isoolefins, (B) at least one isoolefin, and (C) at least one alpha olefin, all having four carbon atoms per molecule, subjecting said feedstock in a catalytic distillation zone to catalytic distillation conditions which favor (1) conversion of said alpha-olefins to additional internal linear olefins and (2) separation of said internal linear olefins on the one hand from said alpha olefins and isoolefins on the other hand, recovering said internal linear olefins from said catalytic distillation zone, introducing ethylene and said recovered internal linear olefins into a metathesis zone which favors the formation of propylene from said ethylene and internal linear olefins, recovering said propylene as a product of the process, recovering said alpha olefins and isoolefins from said catalytic distillation zone as a second mixture separate from said internal linear olefins, introducing said second mixture into a skeletal isomerization zone which favors the conversion of isoolefins at least in part to internal olefins, and recovering from said skeletal isomerization zone at least a third mixture of alpha olefins, internal olefins, and isoolefins, and returning said third mixture to said catalytic distillation zone.

2. The method of claim 1 wherein in said feedstock (A) contains at least in part butene-1, butene-2, and isobutylene, and (B) contains essentially pure isobutylene, said catalytic distillation zone contains at least one double bond isomerization catalyst which promotes the formation of butene-2 from butene-1, butene-2 is recovered as a product from said catalytic distillation zone and introduced into said metathesis zone, said metathesis zone contains at least one catalyst which promotes the disproportionation of ethylene and butene-2 to form propylene, said second mixture is recovered from said catalytic distillation zone separately from said butene-2 product and contains at least in part butene-1 and isobutylene, said skeletal isomerization zone contains at least one skeletal isomerization catalyst that promotes the conversion of isobutylene to a mixture of butene-1 and butene-2, said mixture of butene-1 and butene-2 along with any unconverted isobutylene being recovered from said skeletal isomerization zone as said third mixture, and said third mixture is returned as feed to said catalytic distillation zone.

3. The method of claim 2 wherein said double bond isomerization conditions within said catalytic distillation zone where said double bond isomerization catalyst is located are a temperature of from about 70° F. to about 270° F. and a pressure of from about 20 psig to about 400 psig, and said catalytic distillation conditions are a temperature and pressure range within said distillation zone that encompasses said double bond isomerization conditions and effects a distillation separation between butene-2 on the one hand and butene-1 and isobutylene on the other hand.

4. The method of claim 2 wherein said metathesis zone conditions are a temperature of from about 300° F. to about 800° F., a pressure of from about 200 psig to about 600 psig, and a weight hourly space velocity of from about 1.0 h⁻¹ to about 100 h⁻¹.

5. The method of claim 2 wherein said skeletal isomerization conditions are a temperature of from about 450° F. to about 1200° F., a pressure of from about 0 psig to about 150 psig, and a weight hourly space velocity of from about 1.0 h⁻¹ to about 50 h⁻¹.

6. The method of claim 2 wherein said double isomerization catalyst is at least one of palladium, platinum, nickel, and rhodium carried on an acidic support.

7. The method of claim 2 wherein said metathesis catalyst is at least one of (1) halides, oxides, and carbonyls of at least one of molybdenum, tungsten, rhenium, and magnesium carried on an acidic support, and (2) cobalt molybdate carried on an acidic support.

8. The method of claim 2 wherein said skeletal isomerization catalyst is at least one zeolite having one dimensional pore structures with a pore size ranging from greater than about 0.42 nm and less than about 0.7 nm.

9. The method of claim 1 wherein said feedstock contains, in addition to said first mixture, butadiene, n-butane, isobutane, and hydrogen; in said catalytic distillation zone said n-butane separates with said butene-2 under said distillation conditions and both n-butane and butene-2 are passed to said metathesis zone; said butadiene, isobutane, and hydrogen separate with said isobutylene and butene-1 and in contact with said double bond isomerization catalyst said butadiene is selectively hydrogenated to a mixture of butene-1 and butene-2; isobutane, isobutylene, and unconverted butene-1 are recovered together from said catalytic distillation zone and passed as feed to said skeletal isomerization zone; and a mixture of butene-1, butene-2, isobutane, and isobutylene is recovered from said skeletal isomerization zone for recycle as feed to said catalytic distillation zone.

10. The method of claim 9 wherein a purge stream containing at least one of n-butane, butene-1, and butene-2 is removed from at least one of said skeletal isomerization zone and said metathesis zone and employed in an alkylation zone to form a gasoline grade alkylate of mixed isooctanes.

11. The method of claim 9 wherein in addition to a propylene product, a separate gasoline grade olefin product is recovered from said metathesis zone.

12. The method of claim 1 wherein said feedstock consists essentially of isobutylene.

13. The method of claim 1 wherein said feedstock consists essentially of at least one alpha olefin.
